(19) United States
(12) Patent Application Publication
(10) Pub. No.: US 2023/0310343 A1
(43) Pub. Date: Oct. 5, 2023

(54) Method of Plant-Based Inhalation for the Treatment of Respiratory Viruses

(71) Applicants: Yvette Rose Webb, Hamilton (CA); Clive Alan Webb, Hamilton (CA)

(72) Inventors: Yvette Rose Webb, Hamilton (CA); Clive Alan Webb, Hamilton (CA)

(21) Appl. No.: 18/024,436

(22) PCT Filed: Sep. 2, 2021
(86) PCT No.: PCT/IB2021/000596

§ 371 Date: Mar. 2, 2023

Related U.S. Application Data
(60) Provisional application No. 63/073,723, filed on Sep. 2, 2020.

(51) Int. Cl. A61K 31/015; A61K 9/00; A61K 45/06; A61M 15/08; A61M 11/04

(52) U.S. Cl. CPC A61K 31/015; A61K 9/0043; A61K 9/0073; A61K 45/06; A61M 11/042; A61M 15/08; A61M 2205/8206

(57) ABSTRACT

Pinene compositions are described that may be used to treat respiratory viruses and method of treating a respiratory virus by inhaling an atomized pinene composition. The pinenes composition may comprise an expedient and pinene. The pinene composition comprises pinene in a concentration of greater than 80% by weight of the pinene composition.

The methods of treatment of the respiratory virus includes filling a reservoir bottle of an atomizer with a liquid substance, the liquid substance being comprised of pinene and an excipient and being suitable for atomization, wherein the pinene is in a concentration of greater than 80% by weight of the liquid substance; and inhaling the liquid substance.

### FIELD OF THE INVENTION

The present invention is directed to a method of pinene vaporization which can be used to treat respiratory viruses. Pinene is a terpene, which may reduce viral replication by binding at the N-terminus of the nucleocapsid protein and inhibiting the natural binding of RNA to the nucleocapsid protein. When a person inhales pinene from an atomizer or a vaporizer, the inhalant/vapor may travel via the respiratory system to target cells infected with a respiratory virus. The person may inhale the pinene composition through the nostrils with a nasal inhaler.

### BACKGROUND

Respiratory viruses are the most frequent causative agents of disease in humans, with significant impact on morbidity and mortality worldwide, mainly in children and the elderly. The COVID-19 pandemic of 2020 presented a difficult challenge to people around the world, as society changed, and lives were lost. The novel virus at the center of the pandemic is SARS-CoV-2. This virus belongs to a family of other similar coronaviruses. Very few pharmaceutical treatments have been developed to fight the respiratory viruses and vaccine trials are underway for SARS-CoV-2. Yet, there remains a need for a natural treatment that targets respiratory viruses including SARS-CoV-2 and inhibits viral replication.

### SUMMARY

Embodiments of the present invention a method of plant extract or plant-based inhalation to treat coronavirus or other respiratory infections. The method comprises a person inhaling a composition comprising a pinene composition. The inhaling may be from either 1) a nasal atomizer to inhale a sprayed liquid comprising pinene into his nasopharynx or 2) a vaporizer to heat a pinene substance until it vaporizes and inhaling a vapor nasally into his nasopharynx, upper airways and lungs. In certain embodiment of the present invention, the pinene substance used for the treatment is a liquid substance comprising pinene and an excipient. In certain embodiments of the invention, the atomizer used in treatment comprises a liquid chamber, a capillary tube, atomizer, actuator, and nasal nozzle. In other embodiments of the invention, the vaporizer used in the treatment comprises a battery, substance chamber, heating element, sensor, and nasal piece or nasal adapter. In additional embodiment of the present invention, additional terpenes or other therapeutic agents may be added to the pinene substance.

### DETAILED DESCRIPTION

In early 2020, a pandemic engulfed the world, resulting in millions of infections. The cause of this pandemic was an extremely transmittable and pathogenic virus, SARS-CoV-2 (severe acute respiratory syndrome coronavirus 2). This virus causes COVID-19 disease. Coronaviruses belong to the Coronaviridae family in the Nidovirales order. Corona represents crown-like spikes on the outer surface of the virus; thus, it was named as a coronavirus. Coronaviruses range in size from 65 to 125 nm in diameter and may belong to one of four different subgroups. SARS-CoV-2 belongs to the beta subgroup of coronaviruses and when a human is infected with this virus it may cause acute respiratory distress syndrome, acute lung injury, pulmonary failure, and/or death. SARS-CoV-2, although similar to other viruses (like MERS and the original SARS virus), appears to pose a greater threat to humans because of its higher transmission rate.

Coronaviruses have a common structure; they all have genes in ORF1 regions that encode proteins for viral replication, nucleocapsid, and spike formation. The spikes found on the outer surfaces of these viruses are made from glycoproteins and facilitate attachment and entry into host cells. The receptor binding domain (RBD) is only loosely attached to these viruses. Coronaviruses may recognize different receptors for entry in human cells, including carbohydrates, aminopeptidases, and exopeptidases. The mechanism of entry differs between coronaviruses, but ultimately depend on cellular proteases, like human airway trypsin-like protease (HAT), cathepsins, and transmembrane protease serine 2 (TMPRSS2).

SARS-CoV-2 has the same basic structure as other coronaviruses, containing spike proteins, nucleoproteins, additional polyproteins, and membrane proteins. The membrane proteins of SARS-CoV-2 include RNA polymerase, 3-chymotrypsin-like protease, papain-like protease, helicase, glycoprotein, and other accessory proteins. SARS coronaviruses use angiotensin-converting enzyme 2 (ACE2) as the key receptor for entry in human cells. Although there is some variation in the genome of SARS-CoV-2, four structural proteins and their corresponding genes are always present: spike (S), envelope (E), membrane (M), and nucleocapsid (N). Structural proteins, their assembly, assembly of the virus, and virion egress are ripe targets for possible treatments of coronaviruses, including SARS-CoV-2.

Some interesting research has been conducted on SARS coronaviruses to determine exactly which of its proteins are the most important for viral proliferation. One study looking at SARS-CoV suggested that N, E, and M structural proteins are key molecules in the assembly and egress of the virus. This study tried to create virus-like particles (VLPs) from SARS-CoV, as VLPs are safe models to study the molecular determinants of virion egress. Attempts to produce these VLPs comprised trying different combinations of structural proteins, like M + E, N + M, and N + E, to determine which proteins were necessary for VLP production. The study concluded that all three, M, N, and E structural proteins are needed for VLP produced from SARS coronaviruses. This conclusion made an important distinction between SARS coronaviruses (which require all three structural proteins) and other coronaviruses (which only require two structural proteins). New light was also shed on the mechanism of egress of SARS coronaviruses as researchers were able to fluorescently tag some of the structural proteins and view the assembly of VLPs and their transport through vesicles.

One recent study sought to target SARS-CoV-2 by more precisely identifying its mechanism of entry into human cells. This study confirmed that the ACE2 is receptor targeted by the virus and that the cellular serine protease TMPRSS2 is used by the virus for S protein priming. Without TMPRSS2′s priming of the spike (S) protein, the spike cannot attach to the ACE2 receptor and cellular entry may be inhibited. This study showed that a clinically proven inhibitor of TMPRSS2 (like camostat mesylate) could be effective at defeating the virus. Another important discovery from this study was the similarity between the entry of SARS-CoV and SARS-CoV2 which suggests that antibody responses raised against SARS-CoV could be effective at protecting against SARS-CoV-2 infection.

A 2014 review of the scientific literature regarding the N proteins identified many important features and functions of the protein in viruses, and specifically SARS-CoV. The review noted that the Nucleocapsid (N) protein has been observed in many different viruses and that it is essential in forming the viral capsid. The viral capsid contains all of the genetic information of the virus, without it timely replication and reliable transmission would not occur. This review also noted that in three different viruses (SARS-CoV, IBV, and MHV) the N proteins performs a similar function by packaging the viral genome for viral assembly; in order to package the viral genome, N proteins must allow interactions between nucleic acids and proteins. The localization of the N protein is also important to its understanding. While the N proteins of many coronaviruses differ in their special distribution, N proteins in SARS-CoV are largely distributed to the cytoplasm. Additionally, it is believed that the interaction of the nucleocapsid with envelope proteins drives the incorporation of the nucleocapsid in enveloped viruses, and such protein-protein interactions are critical for viral assembly. The review also noted that the association of N proteins with the ER-Golgi complex may also reflect a role in virus budding. N proteins we also noted to interact with many host cell proteins, including, hCypA, proteasome subunit p42, the B23 phosphoprotein, Smad3, nRNP-A1, the chemokine CXCL16, translation elongation factor-1 alpha, cellular pyruvate kinase protein, and nucleolin. The N structural protein clearly represents a necessary component of coronaviruses that may be targeted with a COVID-19 treatment. Many different treatments for COVID-19 are currently being developed, some of which target the structural proteins of SARS-CoV-2. There exists a need for an effective and safe treatment for respiratory viruses including COVID-19.

Scientists have worked over the last century to create treatments for viral infections, even producing prophylactics and vaccines. Yet, modern research on common botanical compounds suggests that treatments to many viruses may lie right under our noses. Specifically, essential oils have been the subject of great intrigue over past decades, as studies have shown them to be effective treatments for many different viral infections. Essential oils are whole-plant extracts, meaning they are a mixture of many different compounds found naturally in either the whole plant or one component of a plant. Essential oils are named after the plant from which they are derived, and include, but are certainly not limited to, clary sage essential oil, lavender essential oil, rosemary essential oil, black pepper essential oil, pepper mint essential oil, lemon essential oil, and orange essential oil. The process of making essential oils is well known in the art and usually involves distillation or cold pressing of the plant, followed by the addition of carrier oil to the extracted compounds. The compounds contained within each essential oil are numerous, and studies have been conducted on both the essential oils themselves and the individual compounds within essential oils to determine if they may be effective at fighting viruses.

One study that was published in 2010 looked at whether essential oils and oleoresins could be used to treat avian infectious bronchitis virus (IBV). Avian IBV is a type of coronavirus that is found in chickens, posing a threat to food supplies worldwide. Researchers in this study used a blend of oleoresins and essential oils in a liquid emulsion of pharmaceutical grade excipients for in vivo and in vitro tests to determine its effectiveness against IBV. Past studies have suggested that essential oils are virucides because they are lipophilic and able to disrupt the viral membrane. In the in vitro portion of this study, IBV-infected Vero cells were treated with different dilutions of a blend of essential oils and oleoresins. The results showed an overlapping range of dilutions of the blend that proved to be non-toxic for the cells and also able to reduce the IBV in the cells. The in vivo portion of this study treated IBV-infected chickens with the same blend intranasally and by spraying. The results showed that, compared to untreated chickens, treated chickens had much lower levels of IBV and some treated chickens had no detectable IBV. The results also showed that treated chickens were significantly less likely to transmit IBV to non-infected chickens. The study also noted that its findings had implications for other viruses and other hosts, such as humans.

A different study published in 2012 looked at two essential oils, tea tree oil and eucalyptus oil, to determine if they were effective at inactivating airborne influenza virus. By the time of this studies’ publishing, many other in vitro experiments had already showed essential oils to be powerful disinfectants against multiple different viral species. This study had two important experimental features as it tested both, whether tea tree oil and eucalyptus oil were effective at inactivating airborne influenza when the oils were applied to fibers on a filter surface, and whether tea tree oil and eucalyptus oil were effective at inactivating airborne influenza virus when the oils were introduced in aerosol form. The results of the first important experimental feature showed that when tea tree oil was applied to a filter surface, it was able to completely inactivate the virus and viral plaque formation was completely inhibited. Eucalyptus oil, applied using the same method, had a significant antiviral effect, but was not as efficient as tea tree oil. The results of the second important experimental feature showed that tea tree oil, when introduced in aerosol form, was able to inactivate the airborne influenza virus with up to 99.5% efficiency.

While many studies have demonstrated the effectiveness of essential oils against viruses generally, it is important to zero in on specific viruses, specific essential oils, and even individual botanically sourced compounds to create real targeted treatments for viral infections. One study published in 2008 attempted to do just that, by looking at essential oils, including oil from the L. nobilis plant (commonly referred to as bay leaf) to investigate their effect on SARS-CoV and herpes simplex virus 1 (HSV-1). Bay leaf and its oil have been used in many different countries as a folk remedy for a plethora of disease conditions. With the outbreak of SARS in the 2000′s, there was a lack of treatments to combat the SARS-CoV virus, naturally people looked for natural remedies to combat the disease, including essential oils like bay leaf oil. This study utilized gas chromatography to identify the active compounds in bay leaf oil. The results showed the presence of many different terpenes, including ocimene, pinene, cineole, eremanthin, and dehydrocostuslactone. Having identified the active compounds in bay leaf oil, the oil was tested on SARS-CoV-Infected cells. The results showed that bay leaf oil had strong antiviral activity against SARS-CoV, as the concentration of the oil required to inhibit 50% of virus growth was equal to about one-fifth of the concentration of the oil required to reduce growth of the cell by 50%. The discovery that bay leaf oil possessed such strong antiviral potential increases the value of this study’s gas chromatography results, because the cause of the oil’s antiviral effects must be found within the active compounds of the oil.

Essential oil from a single plant species may contain over one hundred different active compounds, a majority of which may be terpenes. For example, cannabis plants contain over 100 different terpenes, including but not limited to, Myrcene, Limonene, Linalool, Caryophyllene, Pinene, Levomenol (Bisabolol), Eucalyptol, Nerolidol, Carene (Delta-3-Carene), Camphene, Borneol, Terpineol, and Geraniol. Terpenes are fragrant and contribute to the aromatic diversity of many plants. As used herein, the term, terpenes, also includes terpenoids. Terpenes are believed to be the active compounds responsible for the antiviral properties of essential oils. Modern research has turned its sights on terpenes to find effective and targeted treatments for viral infections.

One study that was published in 2014 tested the effects of different essential oils and some of their constituent compounds (terpenes) on cells infected with the influenza (H1N1) virus. The study also tested the terpenes and essential oils in two treatment forms, a liquid phase and a vapor phase, noting that past research found liquid phase terpenes and essential oils may be toxic in nasopharyngeal and oral applications. The results of the study revealed that eucalyptus oil was effective in its vapor phase, while cinnamon oil was effective in its liquid phase and only partially effective in its vapor phase; both oils produced significant reductions in viral plaque formation (i.e. inactivation of the virus). One of the tested terpenes, eugenol (the major active compound in cinnamon oil), proved to be effective in its liquid phase as it was able to cause one hundred percent plaque reduction. Another terpene, citronellol was only partially effective in its liquid phase. Both eugenol and citronellol proved to be effective in their vapor phases. Additionally, the study ran assays to determine whether oils and terpenes could inhibit the activity of hemagglutinin and neuraminidase; if either of these viral membrane (M) proteins is inhibited, viral function and dissemination would be significantly decreased. One terpene, pinene (a major active compound in eucalyptus oil) in its vapor phase, was able to inhibit hemagglutinin activity. Cinnamon oil, in its vapor phase, was able to inhibit the activity of both viral membrane proteins. This study also bolstered previous findings about the potential toxicity of liquid phase oils and terpenes, as it noted that a majority of the liquid phase treatments proved cytotoxic to human lung epithelial cells, while vapor phase treatments showed no adverse effects on the same cells.

A team of researchers published their findings in 2010, which tested the effect of essential oils and terpenes on viral multiplication in herpes simplex virus 1 (HSV-1)-infected cells. The results of the study showed that many different terpenes were able to significantly reduce viral multiplication, including, terpinene, pinene, cymene, terpinen-4-ol, terpineol, thymol, citrol, and cineole. The study noted that all of these terpenes may be suitable for drug treatments, except for cineole and thymol, which had low selectivity indexes.

In order to translate generalized findings about the antiviral properties of terpenes into targeted treatments for specific viruses, research must be conducted to determine exactly how specific terpenes are affecting the viruses. A study that was published in 2010 looked specifically at the effect of cineole on infectious bronchitis virus (IBV) to determine how the compound binds with the virus. IBV is a coronavirus, and thus has four structural proteins and their corresponding genes: spike (S), envelope (E), membrane (M), and nucleocapsid (N). Inhibiting production and assembly of any of these structural proteins could prove to stop or significantly reduce viral multiplication. This study tested the effectiveness of cineole, and found it be highly effective at reducing and halting IBV replication at different concentrations. The study also incubated the cells with cineole at different stages during the viral infection cycle to determine when the terpene has a greater effect on the inhibition of IBV. The results showed that cineole is able to inhibit IBV with moderate effectiveness before the virus has entered the cell, but cineole may be fully effective at inhibiting IBV after penetration of the virus into the cell. This study also tested the docking and molecular dynamics of cineole interacting with IBV using computer simulations. The results of the simulation may be the most insightful findings of the study, as the simulation revealed that cineole (more specifically: 1,8-cineole) binds securely to the N-terminus of the N protein. This binding hinders the natural binding of RNA to the N-terminus. The residues of the N-terminus of the N protein being highly conserved among different coronaviruses makes the N terminus a good target for treatments of non-IB coronaviruses.

A very similar study to the last was published in 2011, testing the effect of pinene on infectious bronchitis virus (IBV) to determine how the compound binds with the virus. This study also contained similar results to the last, as assay determined that pinene (both beta-pinene and alpha-pinene) have anti-IBV properties and inhibit viral replication. In regard to timing, this study found that pinene is able to inhibit IBV with moderate effectiveness before the virus has entered the cell, but pinene may be fully effective at inhibiting IBV after penetration of the virus into the cell. This study also found that, like cineole, pinene binds to the N-terminus of the N protein. This binding is stable and results the prevention of viral genomic RNA from binding to the N-terminus of the N protein. Prior research suggests that RNA binding to the N protein also contributes to modulating cell signaling pathway and is essential to viral replication, making the findings of this study all the more impactful.

Even when compounds are found which may have some effect on the process of viral replication in a lab, another hurdle presents itself: the proper administration of the compound in humans. The method of administering a pharmaceutical compound is determinative of whether the compound with be effective at preventing serious viral infection of the patient, and how easily the patient’s body is able to absorb the compound into the proper tissues. Intranasal drug delivery is one method that has been the subject of substantial research in pharmaceutical R&D. Intranasal delivery is unique in that it is non-invasive (the surfaces of the nasal cavity are easily accessible), results in a fast onset (a drug can rapidly reach therapeutic blood levels akin to intravenous administration), and is often associated with less side effects (due to its targeted nature). Drugs delivered intranasally are able to more efficiently effect the central nervous system and cross the blood-brain barrier by utilizing olfactory and trigeminal nerve pathways that originate as olfactory neuro-epithelium in the nasal cavity and terminate in the brain. These pathways have direct access to the brain, thereby bypassing liver metabolism (which is associated with oral administration of drugs), but drug absorption and nasal permeability through mucosa still present a barrier to effective administration. Even when a drug is not targeted to the brain, but rather for the surrounding tissue of the nasal cavity (for example, tissues of the head, neck and chest), the issue of permeability of the mucosa persists. Recent advances in vaccine development due COVID-19, such as the widespread use of mRNA vaccines with lipid nanoparticles, are innovations which may be used to overcome the obstacle of permeability in the nasal cavity. Researchers have proposed multiple ways that absorption of drugs delivered intranasally may be enhanced for administration through pulmonary or nasal routes. These absorption enhancers include the use of surfactants, cyclodextrins, protease inhibitors, tight junction modulators, muco-adhesive polymers, liposomes, and nanoparticles.

Prior art merely establishes the background for the present invention. The cause of the COVID-19 pandemic, SARS-CoV-2, continues to be transmitted, infecting, and otherwise impacting people around the world. Few therapeutics have been developed to treat respiratory viruses in general and vaccine trials are still underway for SARS-CoV-2. The inventors of the present method remained dedicated to developing a natural treatment for respiratory viruses including coronaviruses, and with innovative insight, they developed a method of pinene inhalation that would be able to aid those afflicted by these viruses. Whereas the prior art describes aromatherapy (the vaporization of essential oils to achieve general health outcomes), the compounds used in those treatments are ineffective at handling a novel viral pandemic. Prior art regarding essential oils and terpenes remains in the folk remedy column. By contrast, the present invention pushes the field of terpenes and their health benefits into the column of mainstream medicine. Embodiments of the present invention, generally, comprise a method for the treatment of Coronavirus infections in humans, utilizing vaporized terpenes targeted for that virus. More specifically, embodiments of the present invention comprise a method for treating Coronavirus infections, with terpene vapor, which targets SARS-CoV-2 and similar viruses.

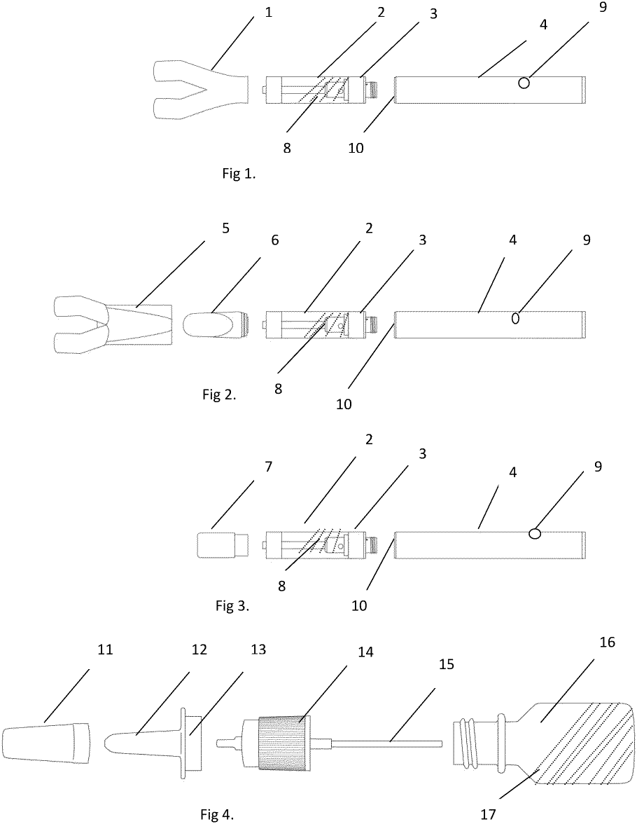

FIG. 1, FIG. 2, and FIG. 3 depict vaporizers which are suitable for use in an embodiment of the invention. FIG. 4 depicts a nasal atomizer device which is suitable for use in an embodiment of the invention. In the preferred embodiment of the invention, a pinene substance (in this embodiment the pinene substance is a liquid substance) comprising pinene and an excipient may be used in the treatment. In the preferred embodiment of the invention, pinene is the primary therapeutic compound, as it may act to inhibit viral genomic RNA binding to the N-terminus of SARS-CoV-2 nucleocapsid proteins; it may also act to inhibit RNA binding to CoV nucleocapsid proteins generally. The excipient may be any excipient known in the art, and includes those excipients used in vaporization of liquid nicotine or cannabis extracts. The modifier primarily acts to ensure that the liquid substance has a viscosity that is suitable for aerosolization in the aerosol pump or vaporization in the vaporizer and may also act as a diluent. The liquid substance may be used to fill the reservoir of the nasal atomizer, or the liquid chamber of a vaporizer. As used herein, fill does not only refer to filling the reservoir of the nasal atomizer or liquid chamber completely, but also refers to inserting some amount of the liquid substance into the reservoir/chamber. Now looking to FIG. 4, in this preferred embodiment of the invention, the nasal atomizer comprises a reservoir bottle (16), a feed tube (15), the spray actuator (14), a nasal spray tip (12), and protective cap (11). The reservoir bottle (16) is connected to the actuator (14) while ensuring the feed tube (15) is seated in the reservoir. The actuator (14) is removable from the reservoir (16) to allow the reservoir (16) to be filled with the liquid substance (17). The nasal spray tip (12) is attached to the other end of the actuator (14). In this embodiment, a user straddles the shoulders (13) of the nasal spray tip (12) with their first and second finger and balances the bottom of the reservoir bottle (16) on the thumb. Pressing the actuator (14) towards the reservoir by pressing down on the shoulders (13) creates pressure forcing the liquid outwards through the actuator (14) and nasal spray tip (13) generating a spray plume. When the actuation pressure is removed, the actuator (14) reseats causing negative pressure in the area of the actuator which pulls liquid from the reservoir (16) through the feed tube (15). The person would inhale nasally once the nasal spray tip (12) was in one of the nares and the actuator (14) pressed down. After a 2 second inhalation, the actuator (14) is released and the person exhales. In this embodiment of the invention, the inhaled atomized pinene travels to the nasopharynx where it remains.

Now looking to FIG. 1, FIG. 2, and FIG. 3, in the alternative preferred embodiment of the invention, the vaporizer comprises a substance chamber (2), nasal piece (1) or nasal adapter (5), or oral piece (6,7), heating element (3), and battery (4). The substance chamber (2) is connected to both the nasal piece (1) or nasal adapter (5) or oral piece (6,7) and battery (4). In this embodiment, the nasal piece (1) or nasal adapter (5) to a mouth piece (6,7), or oral piece (6,7) is removable from the substance chamber (2) to allow the substance chamber to be filled with the pinene substance (8). In this embodiment the pinene substance is a liquid substance, comprising pinene and an excipient. The substance chamber (2) is able to hold 1ml of liquid (8). The heating element (3) is powered by the battery (4) and is able to heat the liquid in the chamber. In this embodiment, the heating element (3) is a heating coil. When the liquid substance within the substance chamber is heated, some of the liquid substance is hot enough to produce vapor which may then be inhaled through the nasal piece, or nasal adapter or mouthpiece. In this alternative embodiment of the invention the vaporizer also comprises a sensor which is able to detect when a person is inhaling from the nasal piece or nasal adapter, or mouthpiece. When the sensor detects inhalation, the heating element is activated via the battery, the liquid substance in the substance chamber is heated, and vapor may be inhaled via the nasal piece or nasal adapter or mouthpiece. The sensor may also stop activating the battery and heating element when inhalation is not detected. In the preferred embodiment of the invention, the battery is a 510 rechargeable battery, and the vaporizer also comprises a port for charging the battery. In this alternative embodiment of the invention, the method for treating respiratory viruses comprises, filling the substance chamber of the vaporizer with 1ml of the liquid substance and attaching the nasal piece or nasal adapter, or mouthpiece to the substance chamber, a person inhaling through the nares via the nasal piece or inhaling through the nasal adapter or through the mouth via the mouthpiece, the sensor detecting the inhalation and activating the battery (it is implied that the battery was previously charged), the battery powering the heating element, the heating element heating the substance chamber, the liquid substance within the substance chamber producing vapor, the person inhaling the vapor through his nose, and the person exhaling the vapor (the person inhaling for 2-5 seconds and exhaling). In this embodiment, the inhaled vaporized pinene travels through the respiratory system, mainly via the nares and nasophamyx, or via the mouth and oropharnyx, to the upper airways and lungs, inhibiting viruses.

Additional embodiments of the present invention differ from the preferred embodiments by utilizing alternate methods and alternate materials for the method. In one embodiment of the invention, the substance chamber of the vaporizer is able to hold only 0.3 ml of liquid. Additional embodiments of the invention allow the substance chamber to hold between 0.3 ml and 1 ml of liquid. Yet, other embodiments allow the substance chamber to hold and vaporize non-liquid substances, such as powders and plant materials. In these embodiments of the invention, the pinene substance used is not a liquid substance, but rather a pinene powder (“powdered pinene”) or pinene plant material. Pinene powder is the powdered form of the terpene pinene, usually included with other compounds to form the powder. Pinene plant material may include cannabis plants with specifically high pinene concentrations, other plants with specifically high pinene concentrations, or other plant materials laced/coated with pinene. In embodiments where the pinene substance is pinene powder or pinene plant material the vapor produced by the vaporizer may be referred to more broadly as the inhalant. In another embodiment of the invention, the battery may not be rechargeable and the vaporizer may not comprise a port for charging the battery. In additional embodiments of the invention, the battery may be rechargeable, but may not be a 510 rechargeable battery, and instead may be another type of battery known in the art that is suitable for the vaporizer. In additional embodiments, the battery may not be rechargeable and may be removable from the other components of the vaporizer, the battery may be rechargeable and may also be removable from the other components of the vaporizer. In embodiments wherein the battery is removable from the vaporizer, the battery may be attached to the vaporizer with a threaded connection or magnet, for example. Additional embodiments of the invention have a different heating element that is suitable for vaporization.

In additional embodiments of the present invention, the pinene substance also comprises additional terpenes or other therapeutic compounds known in the art to treat viral infections. In one such embodiment, the pinene substance also comprises cineole. In another embodiment, the pinene substance also comprises additional therapeutic agents known in the art. In additional embodiments of the present invention the purity of the pinene in the pinene substance may vary. In one such embodiment, the concentration (purity) of the pinene in the pinene substance is greater than 80%; in another embodiment, the concentration of the pinene in the pinene substance is 90-100%. In embodiments of the present invention, the purity of the pinene in the pinene substance is an important factor in the efficacy of the treatment for, Coronaviruses and respiratory viruses generally.

Looking to FIG. 1, FIG. 2, and FIG. 3, in different embodiments of the invention, the vaporizer does not comprise a sensor, but rather comprises a button or switch (9). When the button is pressed or switch is flipped, the battery is activated, allowing the heating element to heat the pinene substance in the substance chamber to produce vapor. The button may be pressed again to deactivate the battery, or the switch may be flipped in the opposite direction to deactivate the battery. In these embodiments, the method for treating respiratory viruses comprises, filling the pinene chamber of the vaporizer with 1ml of the pinene substance (in this embodiment the pinene substance is a liquid substance) and attaching the nasal piece or nasal adapter or mouthpiece to the substance chamber, a person pressing the button or flipping the switch, the battery being activated by the button or switch (it is implied that the battery was previously charged), the battery powering the heating element, the heating element heating the substance chamber, the liquid substance within the substance chamber producing vapor, the person inhaling the vapor through his nostrils with the nasal piece or nasal adapter, or through the mouth via the mouthpiece, the person exhaling the vapor (the person inhaling for 2-5 seconds and exhaling), and the person pressing the button again or flipping the switch in the opposite direction. Now looking to FIG. 1, FIG. 2, and FIG. 3, showing vaporizer devices that may be used in embodiments of the invention. The nasal piece (1) and the nasal adapter (5) and oral piece (6,7) and substance chamber (2) are shown detached from the other components of the vaporizer for visualization purposes only. The button (9) is connected to the battery (4), the charging port (10) is connected to the battery (4), and the heating element (3), is connected to the battery (4). Now looking to FIG. 4., in an embodiment of the invention a nasal atomizer device that may be used. The protective cap (11), nasal spray tip (12), actuator (16), and reservoir bottle (16) are shown detached from the other components of the atomizer for visualization purposes only. The feed tube (15) is connected to the actuator chamber and rests in the reservoir bottle (16).

In yet another embodiment of the present invention, the vaporizer may instead comprise a button that must be held to continue the activation of the heating element via the battery. When the button is released, the battery is deactivated. In yet another embodiment of the invention, the vaporizer device used to create the pinene vapor may not have a mouthpiece, nasal piece, or nasal adapter; the device may emit pinene vapors freely into the air and the person may inhale the vapor without the need for any direct facial contact with the device; and alternatively the device may have yet other ways than previously described to attach to the person’s face, such as through a inhalation feature that cover the mouth and nose. In additional embodiments of the invention, the nasal piece or nasal adaptor for the vaporizer may have two portions (one for each nostril); but in other embodiments, the nasal piece or nasal adaptor for the vaporizer may only have one portion (for one nostril) wherein the inhalant may be inhaled trough only one nostril at a time.

In additional embodiments of the invention, the method comprises the person inhaling or exhaling the inhalant for a different, either longer or shorter, period of time. Inhalation and exhalation may also be performed at specific times and specific frequencies daily. In additional embodiments of the invention, the nasal piece or nasal adapter does not need to be removed to fill the substance chamber, and the substance chamber comprises some other inlet to allow for filling.

The embodiments of the described devices methods are not limited to the embodiments, components, method steps, and materials disclosed herein, as such components, process steps, and materials may vary. Moreover, the terminology employed herein is used to describing exemplary embodiments only and the terminology is not intended to be limiting since the scope of the various embodiments of the present invention will be limited only by the appended claims and equivalents thereof.

Therefore, while embodiments of the invention are described with reference to exemplary embodiments, those skilled in the art will understand that variations and modifications can be affected within the scope of the invention as defined in the appended claims. Accordingly, the scope of the various embodiments of the present invention should not be limited to the above discussed embodiments and should only be defined by the following claims and all equivalents.

1. A method of pinene atomization, using an atomizer device, for the treatment of respiratory viruses comprising:
a. filling a reservoir bottle of an atomizer with a liquid substance, the liquid substance being comprised of pinene and an excipient and being suitable for atomization, wherein the pinene is in a concentration of greater than 80% by weight of the liquid substance;
b. connecting the reservoir bottle to the actuator chamber with a feed tube, the feed tube being attached to the actuator, a nasal spray tip being attached to the actuator chamber, and the nasal spray tip being attached to a protective cap;
c. removing the protective cap, inserting the nasal spray tip in a nostril;
d. inhaling while applying manual force the actuator, the actuator causing the liquid substance to produce aerosol, the aerosol traveling through the nostril; and
e. exhaling.

2. The method of claim 1, wherein the liquid substance also comprises additional terpenes.

3. The method of claim 1, wherein the liquid substance also comprises additional therapeutic compounds.

4. The method of claim 1, wherein the purity of the pinene in the liquid substance is greater than 85%.

5. The method of claim 1, wherein the purity of the pinene in the liquid substance is between 90% and 100%.

6. A method of pinene vaporization for the treatment of respiratory viruses comprising:
a. filling the substance chamber of a vaporizer with a pinene substance, wherein the pinene substance comprises pinene in a concentration greater than 80% by weight of the pinene substance; the vaporizer comprising a battery, substance chamber, sensor, and heating element; the sensor being connected to the battery; the battery being connected to the heating element; the heating element being attached to the substance chamber; the pinene substance being suitable for vaporization;
b. attaching a nasal piece or a nasal adapter that attaches to an existing mouthpiece or an oral piece to the substance chamber of the vaporizer;
c. a person inhaling through the nose, the nasal piece or nasal adapter being in the nares, or inhaling through the mouth, the mouthpiece being in the mouth and exhaling;
d. the sensor detecting the inhalation;
e. the sensor activating the battery;
f. the battery powering the heating element;
g. the heating element heating the pinene substance in the substance chamber;
h. the pinene substance producing vapor; and
i. the person inhaling the vapor through the nares and exhaling the vapor.

7. The method of claim 6, wherein the battery is a 510 rechargeable battery, and the vaporizer also comprises a charging port for the battery, and the 510 rechargeable battery is charged prior to filling the substance chamber with the pinene substance.

8. The method of claim 6, wherein the person inhales the vapor for 2-5 seconds and exhales the vapor.

9. The method of claim 6, wherein the pinene substance is a liquid substance, the liquid substance comprising pinene and an excipient, and wherein the substance chamber is able to hold between 0.3 ml and 1 ml of liquid.

10. The method of claim 6, wherein the pinene substance also comprises additional terpenes.

11. The method of claim 6, wherein the pinene substance also comprises additional therapeutic compounds.

12. The method of claim 6, wherein the heating element is a heating coil.

13. The method of claim 6, wherein the purity of the pinene in the pinene substance is greater than 85%.

14. The method of claim 6, wherein the purity of the pinene in the pinene substance is between 90% and 100%.

15. The method of claim 6, wherein the nasal piece or nasal adaptor consists of only one nasal portion for one nostril.

16. A method of pinene vaporization for the treatment of respiratory viruses comprising:
a. filling the substance chamber of a vaporizer with a pinene substance; the vaporizer comprising a button/switch, battery, substance chamber, and heating element; the button/switch, being connected to the battery; the battery being connected to the heating element; the heating element being attached to the substance chamber, the substance chamber being able to hold at least 1ml of liquid; the pinene substance being suitable for vaporization;
b. attaching a nasal piece or nasal adapter or oral piece to the liquid chamber of the vaporizer;
c. the person pressing the button/ flipping the switch
d. the button/switch activating the battery;
e. the battery powering the heating element;
f. the heating element heating the pinene substance in the substance chamber;
g. the pinene substance producing vapor; and
h. a person inhaling through the nose, the nasal piece or nasal adapter being in the nares or inhaling through the mouth, the mouthpiece being in the mouth and exhaling.

17. The method of claim 16, wherein the battery is a 510 rechargeable battery, and the vaporizer also comprises a charging port for the battery, and the 510 rechargeable battery is charged prior to filling the substance chamber with the pinene substance.

18. The method of claim 16, wherein the person inhales the vapor for 2-5 seconds and exhales the vapor.

19. The method of claim 16, wherein the pinene substance is a liquid substance, the liquid substance comprising pinene and an excipient, and wherein the substance chamber is able to hold between 0.3 ml and 1 ml of liquid.

20. The method of claim 16, wherein the pinene substance also comprises additional terpenes.

21. The method of claim 16, wherein the pinene substance also comprises additional therapeutic compounds.

22. The method of claim 16, wherein the heating element is a heating coil.

23. The method of claim 16, wherein the purity of the pinene in the pinene substance is greater than 80%.

24. The method of claim 16, wherein the purity of the pinene in the pinene substance is between 90% and 100%.

25. The method of claim 16, wherein the nasal piece or nasal adaptor consists of only one nasal portion for one nostril.

26. A method of pinene vaporization for the treatment of Respiratory viruses comprising:
a. filling the substance chamber of a vaporizer with a pinene substance; the vaporizer comprising a battery, substance chamber, nasal piece or nasal adapter or oral piece, sensor, and heating element; the sensor being connected to the battery; the battery being connected to the heating element; the heating element being attached to the substance chamber; the substance chamber being connected to the nasal piece or nasal adapter or oral piece; the pinene substance being suitable for vaporization;
b. a person inhaling through the nose, the nasal piece or nasal adapter being in the nares, or inhaling through the mouth, the mouthpiece being in the mouth and exhaling;
c. the sensor detecting the inhalation;
d. the sensor activating the battery,
e. the battery powering the heating element;
f. the heating element heating the pinene substance in the substance chamber;
g. the pinene substance producing vapor; and
h. the person inhaling the vapor through the nares or the mouth and exhaling the vapor.

27. The method of claim 26, wherein the battery is a 510 rechargeable battery, and the vaporizer also comprises a charging port for the battery, and the 510 rechargeable battery is charged prior to filling the substance chamber with the pinene substance.

28. The method of claim 26, wherein the person inhales the vapor for 2-5 seconds and exhales the vapor.

29. The method of claim 26, wherein the pinene substance is a liquid substance, the liquid substance comprising pinene and an excipient, and wherein the substance chamber is able to hold between 0.3 ml and 1 ml of liquid.

30. The method of claim 26, wherein the pinene substance also comprises additional terpenes.

31. The method of claim 26, wherein the pinene substance also comprises additional therapeutic compounds.

32. The method of claim 26, wherein the heating element is a heating coil.

33. The method of claim 26, wherein the purity of the pinene in the pinene substance is greater than 80%.

34. The method of claim 26, wherein the purity of the pinene in the pinene substance is between 90% and 100°%.

35. The method of claim 26, wherein the nasal piece or nasal adaptor consists of only one portion (for one nostril).

36. A method of pinene vaporization for the treatment of Respiratory viruses comprising:
a. filling the substance chamber of a vaporizer with a pinene substance; the vaporizer comprising a button/switch, battery, liquid chamber, nasal piece or nasal adapter or mouthpiece, and heating element; the button/switch, being connected to the battery; the battery being connected to the heating element; the heating element being attached to the substance chamber, the substance chamber being connected to the nasal piece or nasal adapter or mouthpiece; the pinene substance being suitable for vaporization;
b. the person pressing the button/ flipping the switch
c. the button/switch activating the battery;
d. the battery powering the heating element;
e. the heating element heating the pinene substance in the substance chamber,
f. the pinene substance producing vapor; and
g. the person inhaling the vapor through the nose, the nasal piece or nasal adapter being in the nares, or inhaling through the mouth, the mouthpiece being in the mouth and exhaling.

37. The method of claim 36, wherein the battery is a 510 rechargeable battery and the vaporizer also comprises a charging port for the battery, and the 510 rechargeable battery is charged prior to filling the substance chamber with the pinene substance.

38. The method of claim 36, wherein the person inhales the vapor for 2-5 seconds and exhales the vapor.

39. The method of claim 36, the pinene substance is a liquid substance, the liquid substance comprising pinene and an excipient, and wherein the substance chamber is able to hold between 0.3 ml and 1 ml of liquid.

40. The method of claim 36, wherein the pinene substance also comprises additional terpenes.

41. The method of claim 36, wherein the pinene substance also comprises additional therapeutic compounds.

42. The method of claim 36, wherein the heating element is a heating coil.

43. The method of claim 36, wherein the purity of the pinene in the pinene substance is greater than 80%.

44. The method of claim 36, wherein the purity of the pinene in the pinene substance is between 90% and 100%.

45. The method of claim 36, wherein the nasal piece or nasal adaptor consists of only one nasal portion for one nostril.

46. An atomizer device, which can be used for pinene atomization for the treatment of Respiratory viruses comprising:
a reservoir bottle, a feed tube, a spray actuator, a nasal spray tip, and a protective cap;
the reservoir bottle being able to hold a liquid substance;
the spray actuator being attached to the nasal spray tip and the feed tube, and being reversibly attachable to the reservoir bottle;
the protective cap being reversibly attachable to the nasal spray tip;
the spray actuator, when attached to the reservoir bottle, being able to create pressure when pressed down, that pressure forcing the liquid substance through the feed tube and through the nasal spray tip, generating a spray plume of the liquid substance;
the spray actuator, when pressure is removed, being able to reseat itself on the reservoir bottle, causing negative pressure which pulls the liquid substance from the reservoir bottle through the feed tube.

47. The atomizer device of claim 46, wherein the liquid substance which can be used in the atomizer device comprises pinene and an excipient.

48. The atomizer device of claim 46, wherein the liquid substance which can be used in the atomizer device also comprises additional terpenes.

49. The atomizer device of claim 46, wherein the liquid substance which can be used in the atomizer device also comprises additional therapeutic compounds.

50. The atomizer device of claim 46, wherein the purity of the pinene in the liquid substance is greater than 80%.

51. The atomizer device of claim 46, wherein the purity of the pinene in the liquid substance is between 90% and 100%.
